(19) United States
(12) Patent Application Publication
(10) Pub. No.: US 2010/0197977 A1
(43) Pub. Date: Aug. 5, 2010

(54) Allyl diacetate decomposition

(76) Inventor: Stephen H. Harris, Kennett Square, PA (US)

Correspondence Address: LyondellBasell Industries, 3801 WEST CHESTER PIKE, NEWTOWN SQUARE, PA, 19073, US

(21) Appl. No.: 12/322,650

(22) Filed: Feb. 5, 2009

(51) Int. Cl. C07C 29/09; C09K 3/00

(52) U.S. Cl. 568/877; 252/372

(57) ABSTRACT

A process for purifying an acetoxylation mixture is disclosed. Allyl acetate, water, acetic acid, and from 0.1 to 10 wt. % allyl diacetate are contacted in the vapor phase with a solid acidic catalyst under conditions effective to decompose the allyl diacetate and generate an intermediate stream comprising allyl acetate, water, acetic acid, and acrolein. Acrolein is then removed from the intermediate stream, preferably by distillation, to give an allyl acetate-containing product stream. Usually, this product stream is then hydrolyzed to produce allyl alcohol. The invention includes processes in which propylene first reacts with oxygen and acetic acid in the presence of a noble metal catalyst to generate the acetoxylation mixture.

### FIELD OF THE INVENTION

The invention relates to the manufacture of allyl acetate from the acetoxylation of propylene with oxygen and acetic acid.

### BACKGROUND OF THE INVENTION

Allyl acetate, a valuable intermediate for making allyl alcohol, is available from the reaction of propylene, acetic acid, and oxygen in the presence of a noble metal catalyst, typically palladium. This “acetoxylation” reaction is normally performed in the vapor phase. A heated mixture of the reactants is typically contacted with a bed of supported metal catalyst, and products of the acetoxylation reaction are separated by distillation.

Allyl diacetate, also known as “allylidene diacetate” or 1,1-diacetoxy-2-propene, is an impurity formed during acetoxylation. It is essentially an acetal derived from the reaction of acrolein and two equivalents of acetic acid. Most references that discuss allyl acetate manufacture by acetoxylation, however, are silent regarding the formation or removal of allyl diacetate. Instead, they discuss the catalysts and promoters used for the principal reaction.

Thus, U.S. Pat. Nos. 4,647,690 and 4,571,431, for instance, teach to make allyl acetate by reacting propylene, acetic acid, and oxygen in the presence of palladium, potassium, and bismuth in the presence of an additional rubidium or magnesium promoter. U.S. Pat. No. 7,265,243 similarly teaches an acetoxylation of propylene to allyl acetate. Here, the acetoxylation catalyst activity and lifetime are enhanced by including some copper or gold, a tin promoter, and a small amount of water with the supported palladium catalyst. U.S. Pat. No. 3,925,452 teaches acetoxylation of propylene to make allyl acetate using supported palladium and added water wherein the water:acetic acid ratio is adjusted to allow separation of substantially pure allyl acetate and water phases. U.S. Pat. No. 3,917,676 includes lead and an alkali or alkaline earth metal carboxylate with the palladium acetoxylation catalyst. “Diester” byproducts are mentioned, but no separation from allyl acetate is indicated. Finally, U.S. Pat. No. 5,011,980 describes yet another approach to improving catalyst selectivity and lifetime, but like the other references described in this paragraph, is silent regarding the generation or process for removing allyl diacetate.

Despite these teachings, allyl diacetate is a known acetoxylation impurity, and at least two Japanese companies have looked for ways to remove it. Kazuyuki et al. (Daicel, JP Publ. No. 01-250338), for example, suggest isolating as a sidedraw distillation product a mixture comprising water, acetic acid, acrolein, and allyl diacetate. Allyl diacetate in the sidedraw stream is hydrolyzed (to acrolein and acetic acid) in a separate reactor, and the hydrozylate is returned to the distillation column where acrolein is removed overhead and acetic acid free of allyl diacetate is recovered as a bottom product. Unfortunately, this process is impractical because water, acetic acid, and allyl diacetate are not simultaneously present anywhere in the distillation column.

Naomichi et al. (Kuraray, JP Publ. No. 53-071009) teach to decompose the allyl diacetate produced in an acetoxylation process by simply heating the product mixture. However, our heating of an acetoxylation condensate (containing water, allyl alcohol, allyl acetate, allyl diacetate, and acetic acid) at 120° C. for two hours in a sealed container converted only about half of the diallyl acetate to acrolein and acetic acid. When we heated the mixture in the presence of a small amount of p-toluenesulfonic acid, all of the diallyl acetate was converted to acrolein, but these conditions also hydrolyzed allyl acetate.

Another approach involves distillation to recover the allyl acetate, leaving the allyl diacetate behind as a residue (see Naomichi et al., Kuraray, Jap. Publ. No. 61-238745), followed by treatment of the residue to convert allyl diacetate to acrolein. Unfortunately, that solution is energy-intensive and sacrifices too much of the desired allyl acetate in the distillation residue.

In sum, a better way to remove the allyl diacetate formed during propylene acetoxylation is needed. A preferred approach would avoid the need to remove allyl diacetate either in a sidedraw stream or as a heavy impurity; instead, it would effectively convert most or all of the allyl diacetate to volatile materials that are easy to separate from allyl acetate. Ideally, the method could be practiced commercially in conjunction with the two-step manufacture of allyl alcohol from propylene via acetoxylation and allyl acetate hydrolysis.

### SUMMARY OF THE INVENTION

In one aspect, the invention is a process for purifying an acetoxylation mixture. A mixture comprising allyl acetate, water, acetic acid, and from 0.1 to 10 wt. % of allyl diacetate is contacted in the vapor phase with a solid acidic catalyst under conditions effective to decompose the allyl diacetate and generate an intermediate stream comprising allyl acetate, water, acetic acid, and acrolein. Acrolein is then removed from the intermediate stream to give an allyl acetate-containing product stream, which can be hydrolyzed to produce allyl alcohol. The invention includes processes in which propylene first reacts with oxygen and acetic acid in the presence of a noble metal catalyst to generate the acetoxylation mixture.

### DETAILED DESCRIPTION OF THE INVENTION

An acetoxylation mixture is purified to remove allyl diacetate according one or more processes of the invention. By “acetoxylation mixture,” we mean a mixture comprising allyl acetate, water, acetic acid, and from 0.1 to 10 wt. % of allyl diacetate. Such mixtures are normally obtained by reacting propylene, acetic acid, and oxygen in the presence of a noble metal catalyst under conditions effective to generate allyl acetate, which is the desired end product, along with a minor proportion of allyl diacetate, which is an impurity. In addition to allyl acetate and allyl diacetate, the acetoxylation mixture comprises water, acetic acid, and usually traces of other components. The exact content of the acetoxylation mixture will depend upon the nature of the particular acetoxylation process, the catalyst choice, equipment, reaction conditions, and other factors. However, a typical acetoxylation mixture contains 30-60 wt. % of allyl acetate, 1-3 wt. % of allyl diacetate, 2-10 wt. % water, and 35-65 wt. % of acetic acid.

The acetoxylation mixture is most commonly generated by procedures that are already well known, and are described, for example, in U.S. Pat. Nos. 7,265,243; 5,011,980; 4,647,690; 4,571,431; 3,925,452; and 3,917,676, the teachings of which are incorporated herein by reference. As discussed earlier, a noble metal catalyst, preferably palladium, is used, and the catalyst is advantageously combined with other metals or promoters to increase activity, prolong catalyst lifetime, or enhance conversion and selectivity. One suitable acetoxylation mixture for use in the inventive process is produced by reacting propylene, acetic acid, and oxygen in the presence of palladium supported on alumina and promoted with gold and an alkali metal acetate such as potassium acetate or cesium acetate. Such a catalyst provides a good conversion of propylene to allyl acetate, but the acetoxylation mixture also contains from 1 to 6 wt. % of allyl diacetate.

In the inventive process, an acetoxylation mixture is purified to remove some or all of the allyl diacetate. The allyl diacetate, essentially an acetal, is “decomposed” or converted to one equivalent of acrolein and two equivalents of acetic acid. Acrolein is more volatile than water, acetic acid, or allyl acetate, so it can be removed conveniently from the decomposed product mixture as an overhead distillation cut.

The acetoxylation mixture is contacted in the vapor phase with a solid acidic catalyst under conditions effective to generate an intermediate stream comprising allyl acetate, water, acetic acid, and acrolein. By “vapor phase,” we mean that the acetoxylation mixture is heated or kept hot so that most and preferably all of it, normally a liquid under ambient conditions, is a vapor prior to its exposure to the solid acidic catalyst. Usually, reaction products from the acetoxylation unit are simply transferred while still hot to the allyl diacetate decomposition section.

Acetoxylation mixtures may need to be preheated to vaporize most or all of the liquid. For a typical lab-scale operation, such pre-heating is conveniently accomplished by feeding the liquid acetoxylation mixture to a pre-heat zone containing glass beads or the like for a time sufficient to vaporize most or all of the liquid.

A solid acidic catalyst is used. Suitable solid acidic catalysts are acidic enough to convert at least a portion (preferably all) of the allyl diacetate contained in acetoxylation mixtures to acrolein. However, the solid acidic catalyst should promote allyl diacetate decomposition without also disturbing the desired allyl acetate product. If the solid acidic catalyst is too aggressive, a side reaction can take place in which allyl acetate and acetic acid react to give propylene glycol diacetates; this side reaction is preferably avoided.

Suitable solid acidic catalysts generally include clays; mixed oxides (silica-aluminas, silica-titanias, alumina-borias, silica-zirconias, silica-magnesias, and the like); molecular sieves and zeolites; ion-exchange resins; heteropolyacids; inorganic oxides, sulfates, nitrates, phosphates (e.g., AIPOs and SAPOS), and halides; activated carbons; and the like, and mixtures thereof. Additional suitable solid acidic catalysts are described in U.S. Pat. Nos. 7,344,635 and 5,326,923, the teachings of which are incorporated herein by reference, and in K. Tanabe et al., New Solid Acids and Bases: Their Catalytic Properties, Elsevier, New York (1989). Preferred solid acidic catalysts have relatively low acidity. Silica-aluminas and ammonium or metal-containing Y-zeolites, are particularly preferred. Suitable though less preferred catalysts include the more acidic H-beta and H-Y zeolites, which effectively decompose allyl diacetate but, under at least some conditions, also promote propylene glycol diacetate formation (see Comparative Example 11). The solid acidic catalyst can be used in any desired form or shape, i.e., powder, granules, tablets, extrudates, or the like.

The vaporized acetoxylation mixture is contacted with the solid acidic catalyst under conditions effective to decompose allyl diacetate and produce an intermediate stream comprising allyl acetate, water, acetic acid, and acrolein. Conveniently, effluent from the acetoxylation zone is transferred while hot to the reaction zone for allyl diacetate decomposition. After exposure to the solid acidic catalyst, and conversion of allyl diacetate to acrolein, the products are usually transferred to a distillation tower for separation. Ideally, under the reaction conditions, most or all of the allyl diacetate present in the acetoxylation mixture is converted to acrolein. Typical conversions of allyl diacetate to acrolein range from 50% to 100%, generally at least 75%, and more typically from 85% to 99%.

Any convenient reaction temperature or pressure can be selected. Preferably, the acetoxylation mixture is contacted with the solid acidic catalyst at a temperature within the range of 80° C. to 290° C., more preferably from 100° C. to 250° C., most preferably from 130° C. to 200° C., and at pressures from 0.1 to 100 atm, preferably 0.5 to 10 atm, and most preferably at 1 atm. The feed rate can vary within a wide range, but preferably the gas hourly space velocity (GHSV) is within the range of 500 to 10,000 h⁻¹, more preferably from 3,000 to 6,000 h⁻¹. A carrier gas such as nitrogen or argon is often used to dilute the acetoxylation mixture prior to contacting it with the solid acidic catalyst, since this allows fine adjustment of the GHSV and facilitates heat removal.

The intermediate stream (allyl acetate, water, acetic acid, acrolein, and traces of other components) can be condensed, collected, and saved for further processing later if desired. More economically, however, the hot stream is immediately processed further to remove acrolein. While any desired means of separation can be used, flashing or distillation is most useful because acrolein is more volatile than the other, more-valuable components of the intermediate stream. Thus, the intermediate stream is preferably sent immediately to a distillation process in which the acrolein is removed as an overhead cut. The residue is an allyl acetate-containing product stream that is normally purified to isolate allyl acetate from water and acetic acid. Such purification might be done by water washing or other extractive workup, or the allyl acetate can be isolated by distillation. Distillation is preferred.

Allyl acetate has limited utility as a solvent and monomer. Its greatest use is as an intermediate for making allyl alcohol. Thus, in one aspect, the invention includes processes in which the allyl acetate-containing product stream is hydrolyzed to produce allyl alcohol, a compound used to make pesticides, drugs, and a variety of polymer resins, including CR-39 resin and styrene-allyl alcohol copolymers. This is normally accomplished by reacting the allyl acetate-containing product stream with water in the presence of an acidic catalyst, preferably a sulfonic acid resin (such as Amberlyst 15), according to well-known methods. See, e.g., U.S. Pat. No. 3,970,713, the teachings of which are incorporated herein by reference, Brit. Pat. No. 1,306,219, and U.S. Pat. Appl. Publ. No. 2006/0084829.

The invention includes processes that include a reaction step to generate the acetoxylation mixture. In such a step, the mixture comprising allyl acetate, water, acetic acid, and from 0.1 to 10 wt. % allyl diacetate is generated by reacting propylene, acetic acid, and oxygen in the presence of a noble metal catalyst, preferably palladium. Suitable acetoxylation processes and catalysts useful therein have been thoroughly described elsewhere (see U.S. Pat. Nos. 7,265,243; 5,011,980; 4,647,690; 4,571,431; 3,925,452; and 3,917,676, the teachings of which are incorporated herein by reference).

Thus, for instance, one process of the invention comprises:

(a) reacting propylene, acetic acid, and oxygen in the presence of a noble metal catalyst to produce an acetoxylation mixture comprising allyl acetate, water, acetic acid, and from 0.1 to 10 wt. % of allyl diacetate;

(b) contacting the mixture in the vapor phase with a solid acidic catalyst under conditions effective to decompose allyl diacetate and generate an intermediate stream comprising allyl acetate, water, acetic acid, and acrolein; and

(c) distilling the intermediate stream to remove acrolein as an overhead cut to give an allyl acetate-containing product stream.

The inventive process offers numerous advantages:

1. Conversion to acrolein. Because allyl diacetate is converted to acrolein, a high-boiling impurity (allyl diacetate) is eliminated in favor of a low-boiling one (acrolein). This enables purification of allyl acetate by distillation to remove just a small fraction of low-boiling material as an overhead cut. Absent the inventive process, the skilled person must distill all of the desired allyl acetate to leave the higher-boiling allyl diacetate behind in a residue. Such an alternative is energy-intensive, cost-prohibitive, and sacrifices too much of the valuable allyl acetate product in the residue.

2. Simple to practice. Contacting acetoxylation mixtures in the vapor phase with a solid acidic catalyst under controlled temperatures and pressures is straightforward. No special reagents or equipment are needed.

3. Easy to integrate. The inventive process is easily combined with both the acetoxylation and the allyl acetate hydrolysis steps normally practiced. Effluent from an acetoxylation unit, while still hot, is simply passed over a catalyst bed prior to transfer to the usual distillation scheme. However, now the allyl diacetate does not accumulate as a high-boiling impurity.

4. High conversion. Conditions can be selected to effect essentially quantitative conversion of allyl diacetate to acrolein (see Tables 1 and 2).

5. Catalyst regeneration. Alkaline promoters (e.g., potassium acetate) are commonly used with the noble metal acetoxylation catalyst, and these substances will gradually leach from the noble metal and eventually poison the solid acidic catalyst used to decompose allyl diacetate to acrolein. However, we found that such catalyst poisoning can be reversed by water washing (see Examples 3 and 5, below). When a water wash would be impractical or is preferably postponed, loss of activity from base poisoning can be compensated for by operating the allyl diacetate decomposition at a higher reaction temperature to boost conversion (see Examples 3 and 4).

The following examples merely illustrate the invention. Those skilled in the art will recognize many variations that are within the spirit of the invention and scope of the claims.

### EXAMPLES 1-5

### Vapor-Phase Conversion of Allyl Diacetate to Acrolein Using Na—Y Zeolite

A two-stage, tubular glass reactor equipped with liquid and gas feed inlets, a pre-heat zone, a reaction zone, thermocouples, exit port, and condenser/ collection vessel is used. The pre-heat zone and reaction zone are wrapped with heating tape. The pre-heat zone is packed with 45 cm³ of glass beads. The reaction zone contains 10 cm³ of Na—Y zeolite extrudates (Zeolyst) or silica-alumina extrudates (Grace Davison). The pre-heat zone is kept at 190 to 210° C. to vaporize the liquid feed prior to exposure to the reaction zone. The liquid feed, a simulated acetoxylation mixture of 2 wt. % allyl diacetate and 5 wt. % water in acetic acid, is introduced at 0.5 mL/min., and nitrogen is cofed to achieve the desired gas hourly space velocity (GHSV) target. The reaction bed temperature is maintained at 160-195° C. Vapors exiting the reaction zone are condensed using a dry-ice bath and are analyzed by gas chromatography. Table 1 shows the results using Na-Y zeolite extrudates as the catalyst. Examples 1 and 2 use an untreated catalyst. Conversion to acrolein is high in both examples.

Examples 3 and 4 use Na-Y zeolite extrudates that have been pre-soaked in aqueous cesium acetate solution for 4 h, then dried. The alkali metal acetate is used to simulate the effect of the alkali metal leaching from an acetoxylation catalyst and eventually overloading the decomposition catalyst bed. The acetate blocks acidic sites of the Na-Y zeolite, but it is easily washed off with water (1 h, then dried) to regenerate the original activity (Example 5). Example 4 shows that the activity loss in the alkali metal-poisoned zeolite can also be compensated for by heating it to a higher temperature (see Table 1).

TABLE 1
Vapor-Phase Conversion of Allyl Diacetate to Acrolein
using Na—Y Zeolite

| | | Bed temp | Conversion | |
|---|---|---|---|---|
| Ex. | GHSV (h⁻¹) | (° C.) | (%) | Comment |
| 1 | 5820 | 168 | 97 | untreated catalyst |
| 2 | 5820 | 195 | 100 | untreated catalyst |
| 3 | 3000 | 160 | 58 | CsOAc-treated catalyst |
| 4 | 4300 | 195 | 98 | CsOAc-treated catalyst |
| 5 | 5820 | 168 | 96 | catalyst water-washed |
| | | | | after CsOAc treatment |

### EXAMPLES 6-9

### Vapor-Phase Conversion of Allyl Diacetate to Acrolein Using Silica-Alumina

For Examples 6-9, the procedure of Examples 1-5 is generally followed using silica-alumina extrudates. The results (Table 2) generally parallel those obtained using Na—Y zeolite extrudates.

TABLE 2
Vapor-Phase Conversion of Allyl Diacetate to Acrolein using
Silica-Alumina

| | | Bed temp | Conversion | |
|---|---|---|---|---|
| Ex. | GHSV (h⁻¹) | (° C.) | (%) | Comment |
| 6 | 5820 | 168 | 99 | untreated catalyst |
| 7 | 3000 | 160 | 89 | CsOAc-treated catalyst |
| 8 | 3000 | 195 | 98 | CsOAc-treated catalyst |
| 9 | 5820 | 170 | 98 | catalyst water-washed after |
| | | | | CsOAc treatment |

### EXAMPLE 10

### Formation of Propylene Glycol Diacetate

The procedure of Examples 1-9 is generally followed using a simplified feed mixture consisting of allyl acetate (30 wt. %) in acetic acid. The idea is to test the tendency of the catalyst to form propylene glycol diacetate from mixtures that contain at least allyl acetate and acetic acid. Neither the Na—Y zeolite nor the silica-alumina catalyst used earlier, when tested at 160° C. and multiple flow rates, forms any measurable amount of propylene glycol diacetate (“PG diacetate”). A small amount of PG diacetate is detected with silica-alumina at 190° C.

### COMPARATIVE EXAMPLE 11

### Formation of Propylene Glycol Diacetate

The procedure of Example 10 is generally followed using the same feed mixture of allyl acetate (30 wt. %) in acetic acid to test other zeolite catalysts. PG diacetate (about 0.5 wt. %) forms using each of H-Y zeolite and H-beta zeolite when tested at 160° C.; this corresponds to about a 1 % yield loss of allyl acetate.

### EXAMPLE 12

### Vapor-Phase Decomposition of Allyl Diacetate in an Acetoxylation Mixture

The procedure of Examples 1-9 is generally followed using a liquid reaction product from an actual acetoxylation unit. The product contains allyl diacetate (2 wt. %) and allyl acetate (50 wt. %) in addition to acetic acid, water, propionaldehyde, and other trace components. Conversion of allyl diacetate (and 1,3-diacetate isomers) to acrolein is near quantitative over both the Na—Y zeolite and the silica-alumina at 160° C. and GHSV=5820 h⁻¹. No PG diacetate is detected.

The examples are meant only as illustrations. The following claims define the invention.

I claim:

1. A process for purifying an acetoxylation mixture, comprising:
(a) contacting a mixture comprising allyl acetate, water, acetic acid, and from 0.1 to 10 wt. % of allyl diacetate in the vapor phase with a solid acidic catalyst under conditions effective to decompose the allyl diacetate and generate an intermediate stream comprising allyl acetate, water, acetic acid, and acrolein; and
(b) removing acrolein from the intermediate stream to give an allyl acetate-containing product stream.

2. The process of claim 1 wherein the catalyst is selected from the group consisting of clays; mixed oxides; molecular sieves and zeolites; ion-exchange resins; heteropolyacids; inorganic oxides, sulfates, nitrates, phosphates, and halides; activated carbons; and mixtures thereof.

3. The process of claim 2 wherein the catalyst is an ammonium or alkali metal-containing Y-zeolite.

4. The process of claim 2 wherein the catalyst is a silica-alumina.

5. The process of claim 1 wherein the mixture is contacted with the catalyst at a temperature within the range of 100° C. to 250° C.

6. The process of claim 1 wherein the mixture is contacted with the catalyst at a gas hourly space velocity within the range of 500 h⁻¹ to 10,000 h⁻¹.

7. The process of claim 1 wherein conversion of allyl diacetate to acrolein and acetic acid is at least 75%.

8. The process of claim 1 wherein acrolein is removed from the intermediate stream by distillation.

9. The process of claim 1 further comprising hydrolyzing the allyl acetate-containing product stream to produce allyl alcohol.

10. A process which comprises:
(a) reacting propylene, acetic acid, and oxygen in the presence of a noble metal catalyst to produce an acetoxylation mixture comprising allyl acetate, water, acetic acid, and from 0.1 to 10 wt. % of allyl diacetate;
(b) contacting the mixture in the vapor phase with a solid acidic catalyst under conditions effective to decompose allyl diacetate and generate an intermediate stream comprising allyl acetate, water, acetic acid, and acrolein; and
(c) distilling the intermediate stream to remove acrolein as an overhead cut to give an allyl acetate-containing product stream.

11. The process of claim 10 wherein step (a) is performed in the presence of an alkaline promoter.

12. The process of claim 11 wherein the solid acid catalyst, during and/or after use in the process, is water washed to maintain high conversions of allyl diacetate to acrolein.

13. The process of claim 11 wherein the solid acid catalyst, during and/or after use in the process, is heated to an increased temperature to maintain high conversions of allyl diacetate to acrolein.

14. The process of claim 10 further comprising hydrolyzing the allyl acetate-containing product stream to produce allyl alcohol.
